(19) United States
(12) Patent Application Publication
(10) Pub. No.: US 2007/0249588 A1
(43) Pub. Date: Oct. 25, 2007

(54) Nicotinic Acetylcholine Receptor Ligands

(75) Inventors: Glen Ernst, Wilmington, DE (US); William Frietze, Wilmington, DE (US); Robert Jacobs, Research Triangle Park, NC (US); Eifion Phillips, Boothwyn, PA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/583,576

(22) PCT Filed: Dec. 20, 2004
(86) PCT No.: PCT/SE04/01941

§ 371 Date: Mar. 26, 2007

Related U.S. Application Data
(60) Provisional application No. 60/531,710, filed on Dec. 22, 2003.

(51) Int. Cl. A61K 31/551; A61P 25/18; A61P 25/24

(52) U.S. Cl. 514/218; 540/575

(57) ABSTRACT

Acetylcholine receptor ligands of formula (I), wherein D, Ar1, E and Ar2 are as described in the specification, diastereoisomers, enantiomers, pharmaceutically-acceptable salts, methods of making, pharmaceutical compositions containing and methods for using the same.

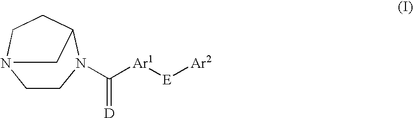

### FIELD OF THE INVENTION

This invention relates to diazabicyclo-octyl amides or pharmaceutically-acceptable salts thereof, processes for preparing them, pharmaceutical compositions containing them and their use in therapy. The invention also relates to compounds that are ligands for nicotinic acetylcholine receptors (nAChRs).

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### DESCRIPTION OF THE INVENTION

This invention concerns nicotinic acetylcholine receptor-active compounds of formula I: 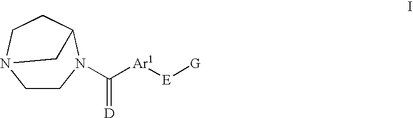
wherein:

D is selected from oxygen, sulfur or N(R¹)₂;

Ar¹ is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

E is a single bond, —O, —S, or —NR²;

G is selected from hydrogen, C₁-C₄alkoxy or Ar², where Ar² is a 5- or 6-membered aromatic or heteroaromatic ring having 0, 1 or 2 nitrogen atoms, 0 or 1 oxygen atoms, and 0 or 1 sulfur atoms;

where each Ar¹ or Ar² moiety independently is unsubstituted or has 1, 2 or 3 substituents selected from —R³, —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;

R¹, R² and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl.

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compounds of the invention are those according to formula I: 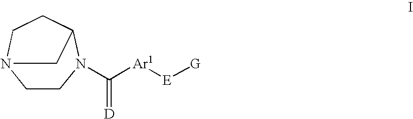
wherein:

D is selected from oxygen, sulfur or N(R¹)₂;

Ar¹ is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

E is a single bond, —O, —S, or —NR²;

G is selected from hydrogen, C₁-C₄alkoxy or Ar², where Ar² is a 5- or 6-membered aromatic or heteroaromatic ring having 0, 1 or 2 nitrogen atoms, 0 or 1 oxygen atoms, and 0 or 1 sulfur atoms;

where each Ar¹ or Ar² moiety independently is unsubstituted or has 1, 2 or 3 substituents selected from —R³, —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;

R¹, R¹ and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or

R¹ and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl,

and stereoisomers, enantiomers, in vivo-hydrolysable precursors and pharmaceutically-acceptable salts thereof.

Particular compounds are those of formula I wherein:

D is oxygen;

Ar¹ is selected from phenyl or a 5-membered heteroaromatic ring having 0 or 1 nitrogen atoms, 0 or 1 oxygen atoms, and 0 or 1 sulfur atoms, or selected from a 9-membered fused aromatic or heteroaromatic ring system having 0, 1, 2 or 3 nitrogen atoms, 0 or 1 oxygen atoms, and 0 or 1 sulfur atoms;

wherein:

E is a single bond;

G is selected from hydrogen, methoxy or Ar², where Ar² is selected from a 6-membered aromatic or heteroaromatic ring having 0 or 1 nitrogen atoms, 0 or 1 oxygen atoms, and 0 or 1 sulfur atoms;

where each Ar¹ or Ar² moiety independently is unsubstituted or has 1, 2 or 3 substituents selected from halogen, —CN, —NO₂, —CF₃, —CH₃ or —C₂H₅;

and stereoisomers, enantiomers, in vivo-hydrolysable precursors and pharmaceutically-acceptable salts thereof.

More particular compounds are those of formula I wherein:

D is oxygen;

Ar¹ is selected from phenyl, furanyl, thiophenyl or 1-methyl-1H-pyrrolyl:

E is a single bond;

G is selected from hydrogen, methoxy, phenyl or pyridyl, and

Ar¹ bears 1 halogen substituent;

and stereoisomers, enantiomers, in vivo-hydrolysable precursors and pharmaceutically-acceptable salts thereof.

Other particular compounds of the invention include those of formula I wherein E represents a single bond; or an enantiomer thereof, and pharmaceutically-acceptable salts thereof.

Still other particular compounds of the invention are those of formula I wherein Ar¹ is furanyl, oxazole or thiophenyl having optional substituents as defined herein.

Particular compounds of the invention are those described herein and pharmaceutically-acceptable salts thereof.

In a further aspect the invention encompasses compounds according to formula I wherein one or more of the atoms is a radioisotope of the same element. In a particular form of this aspect of the invention the compound of formula I is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of the α7 nicotinic acetylcholine receptor. Such tritium-labeled compounds may be used in assays that measure the displacement of a such compounds to assess the binding of ligand that bind to α7 nicotinic acetylcholine receptors.

In another aspect the invention relates to compounds according to formula I and their use in therapy and to compositions containing them.

In another aspect the invention encompasses the use of compounds according to formula I for the therapy of diseases mediated through the action of nicotinic acetylcholine receptors. A more particular aspect of the invention relates to the use of compounds of formula I for the therapy of diseases mediated through the action of α7 nicotinic acetylcholine receptors.

Another aspect of the invention encompasses a method of treatment or prophylaxis of diseases or conditions in which activation of the α7 nicotinic receptor is beneficial which method comprises administering a therapeutically-effective amount of a compound of the invention to a subject suffering from said disease or condition.

One embodiment of this aspect of the invention is a method of treatment or prophylaxis, wherein the disorder is anxiety, schizophrenia, mania or manic depression.

Another embodiment of this aspect of the invention is a method of treatment or prophylaxis of neurological disorders, psychotic disorders or intellectual impairment disorders, which comprises administering a therapeutically effective amount of a compound of the invention.

Another embodiment of this aspect of the invention is a method of treatment or prophylaxis, wherein the disorder is Alzheimer's disease, learning deficit, cognition deficit, attention deficit, memory loss, or Attention Deficit Hyperactivity Disorder.

Another embodiment of this aspect of the invention is a method of treatment or prophylaxis, wherein the disorder is Parkinson's disease, Huntington's disease, Tourette's syndrome, or neurodegenerative disorders in which there is loss of cholinergic synapses.

Another embodiment of this aspect of the invention is a method of treatment or prophylaxis of jetlag, nicotine addiction, craving, pain, and for ulcerative colitis, which comprises administering a therapeutically effective amount of a compound of the invention.

Yet another embodiment of this aspect of the invention is a method for inducing the cessation of smoking which comprises administering an effective amount of a compound of the invention.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition, and pharmaceutically-acceptable additives carrier.

Another embodiment of this aspect of the invention relates to use of a pharmaceutical composition of the invention for the treatment, amelioration or prophylaxis of human diseases or conditions in which activation of the α7 nicotinic receptor is beneficial.

Another embodiment of this aspect of the invention is the use of the pharmaceutical composition of the invention for the treatment or prophylaxis of neurological disorders, psychotic disorders or intellectual impairment disorders.

Another embodiment of this aspect of the invention is the use of the pharmaceutical composition of the invention for the treatment or prophylaxis of Alzheimer's disease, learning deficit, cognition deficit, attention deficit, memory loss, Attention Deficit Hyperactivity Disorder, anxiety, schizophrenia, or mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, craving, pain, and for ulcerative colitis.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

Another embodiment of this aspect of the invention is the use of a compound of the invention in the manufacture of a medicament for the treatment or prophylaxis of human diseases or conditions in which activation of the α7 nicotinic receptor is beneficial.

Another embodiment of this aspect of the invention is the use of a compound of the invention in the manufacture of a medicament for the treatment or prophylaxis of neurological disorders, psychotic disorders or intellectual impairment disorders.

Another embodiment of this aspect of the invention is the use of a compound of the invention in the manufacture of a medicament for treatment or prophylaxis of Alzheimer's disease, learning deficit, cognition deficit, attention deficit, memory loss or Attention Deficit Hyperactivity Disorder.

Another embodiment of this aspect of the invention is the use of a compound of the invention in the manufacture of a medicament for treatment or prophylaxis of anxiety, schizophrenia, or mania or manic depression.

Another embodiment of this aspect of the invention is the use of a compound of the invention in the manufacture of a medicament for treatment or prophylaxis of Parkinson's disease, Huntington's disease, Tourette's syndrome, or neurodegenerative disorders in which there is loss of cholinergic synapses.

Another embodiment of this aspect of the invention is the use of a compound as described above in the manufacture of a medicament for the treatment or prophylaxis of jetlag, pain, or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

For the uses, methods, medicaments and pharmaceutical compositions mentioned herein the amount of compound used and the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

The compounds of formula I, an enantiomer thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier. Examples of diluents, lubricants and carriers are
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing the ingredients.

Compounds according to the invention are agonists of nicotinic acetylcholine receptors. While not being limited by theory, it is believed that agonists of the α7 nicotinic acetylcholine receptor (nAChR) subtype are useful in the treatment or prophylaxis of neurological disorders, psychotic disorders and intellectual impairment disorders, and to have advantages over compounds which are or are also agonists of the α4 nAChR subtype. Therefore, compounds which are selective for the α7 nAChR subtype are preferred. The compounds of the invention are indicated as pharmaceuticals, in particular in the treatment or prophylaxis of neurological disorders, psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain, chronic pain, and in the treatment or prophylaxis of Parkinson's disease, Huntington's disease, Tourette's syndrome, and neurodegenerative disorders in which there is loss of cholinergic synapses.

Compounds of the invention may further useful for the treatment or prophylaxis of jetlag, for use in inducing the cessation of smoking, craving, and for the treatment or prophylaxis of nicotine addiction including that resulting from exposure to products containing nicotine.

It is also believed that compounds according to the invention are useful in the treatment and prophylaxis of ulcerative colitis.

The compounds of the invention have the advantage that they may be less toxic, be more efficacious, be longer acting, have a broader range of activity, be more potent, produce fewer side effects, are more easily absorbed or have other useful pharmacological properties.

The compounds of formula I exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄ alkyl groups include the cyclic alkyl moieties cyclopropyl and cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from: halogen, C₁₋₄alkyl, C₂₋₄alkenyl, C₂₋₄alkynyl, C₁₋₄alkyl, CN, NO₂, and CF₃.

As used herein, unless otherwise indicated, heteroaryl refers to a 5- or 6-membered aromatic or heteroaromatic ring having 1, 2 or 3 heteroatoms selected from nitrogen oxygen and sulfur, provided that heteroaromatic rings contains at least one nitrogen, oxygen, or sulfur atom.

As used herein, unless otherwise indicated, halogen refers to fluorine, chlorine, bromine, or iodine.

Where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of formula I exist in tautomeric or enantiomeric forms, all of which are included within the scope of the invention. The various optical isomers may be isolated by separation of a racemic mixture of the compounds using conventional techniques, e.g. fractional crystallisation, or chiral HPLC. Alternatively the individual enantiomers may be made by reaction of the appropriate optically active starting materials under reaction conditions which will not cause racemisation.

Pharmacology

The pharmacological activity of the compounds of the invention may be measured in the tests set out below:

Test A—Assay for Affinity at α₇ nAChR Subtype

¹²⁵I-α-Bungarotoxin (BTX) Binding to Rat Hippocampal Membranes.

Rat hippocampi are homogenized in 20 volumes of cold homogenisation buffer (HB: concentrations of constituents (mM): tris(hydroxymethyl)aminomethane 50; MgCl₂ 1; NaCl-120; KCl 5: pH 7.4). The homogenate as centrifuged for 5 minutes at 1000×g, the supernatant saved and the pellet re-extracted. The pooled supernatants are centrifuged for 20 minutes at 12000×g, washed, and re-suspended in HB. Membranes (30-80 μg) are incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)] for 2 hours at 21° C., and then filtered and washed 4 times over Whatman glass fiber filters (thickness C) using a Brandel cell harvester. Pre-treating the filters for 3 hours with 1% (BSA/0.01% PEI (polyethyleneimine) in water is critical for low filter blanks (0.07% of total counts per minute). Non-specific binding is described by 100 μM (−)-nicotine, and specific binding is typically 75%.

Test B—Assay for Affinity to the α₄ nAChR subtype

[³H]-(−)-Nicotine Binding.

Using a procedure modified from Martino-Barrows and Kellar (Mol Pharm (1987) 31:169-174), rat brain (cortex and hippocampus) is homogenised as in the [¹²⁵I]α-BTX binding assay, centrifuged for 20 minutes at 12,000×g, washed twice, and then re-suspended in HB containing 100 μM diisopropyl fluorophosphate. After 20 minutes at 4° C., membranes (approximately 0.5 mg) are incubated with 3 nM [³H]-(−)-nicotine, test drug, 1 μM atropine, and either 2 mM CaCl₂ or 0.5 mM EGTA for 1 hour at 4° C., and then filtered over Whatman glass fiber filters (thickness C) (pre-treated for 1 hour with 0.5% PEI) using a Brandel cell harvester. Non-specific binding is described by 100 μM carbachol, and specific binding is typically 84%.

Binding Data Analysis for Tests A and B

IC50 values and pseudo Hill coefficients (n_{H}) are calculated using the non-linear curve fitting program ALLFIT (DeLean A, Munson P J and Rodbard D (1977) Am. J. Physiol., 235:E97-E102). Saturation curves are fitted to a one site model, using the non-linear regression program ENZFITTER (Leatherbarrow, R. J. (1987)), yielding K_{D} values of 1.67 and 1.70 nM for the ¹²⁵I-α-BTX and [³H]-(−)-nicotine ligands respectively. Kᵢ values are estimated using the general Cheng-Prusoff equation:
Kᵢ=IC₅₀/((2+([ligand]/K_{D})ⁿ)^{1/n}−1)
where a value of n=1 is used whenever n_{H}<1.5 and a value of n=2 is used when n_{H}≧1.5. Samples are assayed in triplicate and were typically ±5%. Kᵢ values are determined using 6 or more drug concentrations. The compounds of the invention are compounds with binding affinities (Kᵢ) of less than 10 μM in either Test A or Test B, indicating that they are expected to have useful therapeutic activity.

The compounds of the invention have the advantage that they may be less toxic, be more efficacious, be longer acting, have a broader range of activity, be more potent, produce fewer side effects, are more easily absorbed or have other useful pharmacological properties.

The invention will now be illustrated by the following Examples in which, generally:

(i) operations were carried out at ambient temperature, i.e. in the range 17 to 25° C. and under an atmosphere of an inert gas such as argon or nitrogen unless otherwise stated;

(ii) evaporations were carried out by rotary evaporation in vacuo and work-up procedures were carried out after removal of residual solids by filtration;

(iii) column chromatography (by the flash procedure) and medium pressure liquid chromatography (MPLC) were performed on ICN Ecochrom 60 Angstrom silica gel. In cases where Reverse Phase High Pressure Liquid Chromatography (RP-HPLC) was employed as a method of purification, Gilson instrumentation (215 Injector, 333 Pumps and 155 UV/V is Detector) and a Varian C8 reverse phase column (60 Angstrom irregular load in 8 μm particle size, 41.4 mm ID×250 mm) were employed. Gradient elution was performed with aqueous 0.1% trifluoroacetic acid/acetonitrile with 0.1% trifluoroacetic acid. Sample collection was based on signal at 254 nm unless otherwise noted. In cases where Normal Phase High Pressure Liquid Chromatography (NP-HPLC) was required, Dynamax instrumentation (Dual SD-1 Pumps and UV-1 UV/Vis Detector with a Superprep Flow Cell and a Rainin silica normal phase column (60 Angstrom irregular load in 8 μm particle size, 41.4 mm ID×250 mm) were employed. Isocratic elution was performed with 0.5% isopropyl alcohol in hexanes. Supercritical Fluid Chromatography (SFC) was performed on a Berger Autoprep SFC system generally using methanol (containing 0.5% dimethyl ethyl amine) in carbon dioxide and a Berger Diol column (5 micron, 60 Å pore size).

(iv) yields, where present, are not necessarily the maximum attainable;

(v) in general, the structures of the end-products of the Formula I were confirmed by nuclear magnetic resonance (NMR) and/or mass spectral (MS) techniques; AP/CI mass spectral data were obtained using a Waters Platform LCZ spectrometer and, where appropriate, either positive ion data or negative ion data were collected; NMR chemical shift values were measured on the delta scale proton magnetic resonance spectra were determined using a Bruker Avance 300 spectrometer operating at a field strength of 300 MHz; the following abbreviations have been used: s, singlet; d, doublet; t, triplet; q, quartet; m, multiplet; br, broad;

(vi) intermediates were not necessarily fully purified but their structures and purity were assessed by thin layer chromatographic, HPLC, infra-red (IR) and/or NMR analysis;

(vii) melting points are uncorrected and were determined using a Meltemp 3.0 melting point apparatus or an oil-bath apparatus; melting points for the end-products of the Formula I were determined after crystallization from an appropriate organic solvent or solvent mixture;

(viii) the following abbreviations have been used:

DMF N,N-dimethylformamide

DMSO dimethylsulphoxide

THF tetrahydrofuran

DMA N,N-dimethylacetamide

DCM dichloromethane

Starting Materials and Intermediates

Starting materials are either commercially available or are readily prepared by standard methods from known materials. The following reactions illustrate, but do not limit, the preparation of intermediates.

Intermediates

### Intermediate 1: 1,4-Diazabicyclo[3.2.1]octane

### a) 3-Oxo-piperazin-2-yl-acetic acid ethyl ester

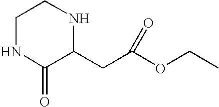

3-Oxo-piperazin-2-yl-acetic acid ethyl ester was prepared according to the procedure described by S. Gubert, et. al. (J. Het. Chem., 30, 1993, 275-276.

### b) 2-Piperazin-2-yl-ethanol

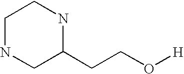

To a mixture of 3-oxo-piperazin-2-yl-acetic acid ethyl ester (2.0 g, 10.74 mmol) in 50 mL of dry THF cooled in an ice bath, was added LAH (1M solution in THF, 20.0 mL, 20.0 mmol) dropwise with stirring under N₂. When addition was complete (c. 10 min), the reaction mixture was refluxed for 3½ h, then cooled in an ice bath. Water (5 mL) was cautiously added with stirring. After stirring for ½ h, the mixture was filtered through a fritted funnel and the collected salts were washed with hot EtOH. The filtrates were combined, dried over MgSO₄, filtered and solvents removed in vacuo. The residue was treated with hot CHCl₃, filtered and the CHCl₃ was evaporated to give a pale yellow oil. The product was obtained in quantitative yield and carried forward without further purification. ¹H NMR (300.132 MHz, CDCl₃) δ 3.82-3.78 (m, 1H), 2.98-2.63 (m, 5H), 2.45-2.36 (m, 1H), 1.62-1.53 (m, 3H), 1.66 (bs, 2H), 1.13 (bs, 1H).

### c) 1,4-Diazabicyclo[3.2.1]octane dihydrochloride salt

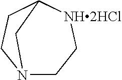

The title compound, 1,4-diazabicyclo[3.2.1]octane was prepared as a dihydrochloride salt from 2-piperazin-2-yl-ethanol according to the procedure described by P. A. Sturn et. al. (J. Med. Chem., 20 (10), 1977, 1333-1337.

### Intermediate 2: (R)-1,4-Diazabicyclo[3.2.1]octane dihydrochloride

### a) ((R)-4-Benzyl-3,6-dioxo-piperazin-2-yl)-acetic acid benzyl ester

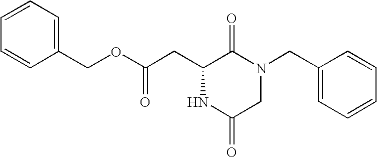

To a cooled (ice bath) solution of dicyclohexylcarbodiimide (3.19 g, 15.46 mmol) in 75 mL of CH₂Cl₂ was added BOC-D-aspartic acid 4-benzyl ester (5 g, 15.46 mmol). The resulting slurry was stirred for 5 min, then N-benzyl glycine ethyl ester (2.9 mL, 15.46 mmol) was added. The suspension was stirred at <5° C. for 2 h, then stirred at room temperature overnight. The reaction mixture was filtered to remove the precipitated dicyclohexylurea. The filter cake washed with a small amount of CH₂Cl₂. The filtrate was evaporated to a viscous oil which was dissolved in diethyl ether and allowed to stand at room temperature for 2 h. The additional precipitate that formed was removed by filtration and the filtrate was concentrated in vacuo to give quantitative yield of a pale yellow viscous oil. ¹H-NMR: 300 MHz, CDCl₃ δ 7.4-7.2 (m, 10H); 5.45 (m, 1H); 5.13 (d, 2H); 4.9-4.5 (m, 2H); 4.3-3.82 (m, 4H); 2.88-2.7 (m, 2H); 1.42, 1.35 (2 s, 9H); 1.23 (m, 3H).

The oil was dissolved in CH₂Cl₂ (20 mL) and trifluoroacetic acid (15 mL) was added. The solution was stirred at room temperature for 2 h, then concentrated in vacuo. The residue was partitioned between EtOAc and saturated aqueous NaHCO₃. The layers were separated and the aqueous phase was back-extracted with EtOAc. The combined organic extracts were dried over MgSO₄, filtered and concentrated in vacuo. 5.1 g of a white solid was obtained (94%). ¹H-NMR: 300 MHz, CDCl₃ δ 7.4-7.2 (m, 10H); 6.46 (br s, 1H); 5.15 (s, 2H); 4.57 (AB quart, 2H); 4.43 (br d, 1H); 3.84 (s, 2H); 3.2-3.13 (m, 1H); 2.91-2.82 (m, 1H).

### b) 2-((R)-4-Benzyl-piperazin-2-yl)-ethanol

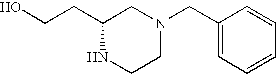

A solution of 5.1 g (14.47 mmol) of ((R)-4-benzyl-3,6-dioxo-piperazin-2-yl)-acetic acid benzyl ester in 60 mL of dry THF was cautiously added to a reaction flask containing 60 mL of 1 M lithium aluminum hydride in THF stirring under N₂. When the addition was complete, the reaction mixture was heated at reflux for 5 h, then kept at 55-60° C. overnight, then refluxed for 7 h, then stirred at room temperature overnight. 15 mL of water was cautiously added with vigorous stirring, then the mixture was stirred for 0.5 h. The resulting slurry was vacuum filtered through a fritted glass funnel and the solids were washed with THF and MeOH. The filtrate was concentrated in vacuo and the residue was taken up in CHCl₃ and extracted twice with 50 mL of 1 N HCl. The aqueous extracts were combined and washed twice with CHCl₃. The aqueous phase was made basic by addition of a solution of 5 g of NaOH in 50 mL of water. The resulting cloudy alkaline aqueous mixture was extracted twice with 50 mL CHCl₃. These organic extracts were combined, dried over MgSO₄, filtered and concentrated in vacuo to give 2.87 g of a colorless oil that slowly solidifies (90%). ¹H-NMR: 300 MHz, CDCl₃ δ 7.4-7.2 (m, 5H); 3.79 (m, 2H); 3.48 (s, 2H); 3.02-2.78 (m, 3H); 2.77-2.68 (m, 2H); 2.02 (m, 1H); 1.84 (m, 1H); 1.58 (m, 2H).

### c) (R)-2-Piperazin-2-yl-ethanol

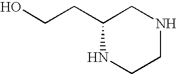

A Parr bottle was charged with a solution of 2-((R)-4-benzyl-piperazin-2-yl)-ethanol (2.87 g, 13.03 mmol) in 50 mL of MeOH. 500 mg of Pearlman's catalyst was added and the mixture was placed under 50 psi of H₂ and agitated on a Parr shaker. After 1 hr, a large initial uptake of H₂ was observed. The vessel was repressurized to 50 psi and agitated overnight. The bottle was purged of H₂ and removed from the Parr shaker. The reaction mixture was filtered through diatomaceous earth and the filter cake washed with MeOH. The filtrate was concentrated in vacuo to give quantitative yield of product. ¹H-NMR: 300 MHz, CDCl₃ δ 3.82 (m, 2H); 3.02-2.69 (m, 6H); 2.6-2.52 (m, 1H); 1.62 (m, 2H).

### d) (R)-2-(2-Chloro-ethyl)-piperazine dihydrochloride

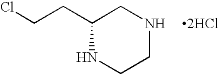

20 mL of thionyl chloride were cautiously added to a chilled (ice bath) flask containing (R)-2-piperazin-2-yl-ethanol (approx. 13.03 mmol). The reaction mixture was cautiously heated to 80° C. and stirred at this temperature for 2 h. At this point, the volume of SOCl₂ was reduced in vacuo. The resulting residue was treated cautiously with water until a solution resulted. This solution was reduced in volume in vacuo to remove volatile by-products. The residue was redissolved in a minimal amount of water and decolorizing carbon was added. The aqueous mixture was heated at 80° C. for 15 min, then vacuum filtered through a fritted glass funnel. Acetone was added to the pale yellow filtrate to precipitate the product. The precipitate was collected by vacuum filtration and washed with acetone. More acetone was added to the filtrate to give another crop of precipitate. In this manner, 1.47 g of a white solid was collected from 3 crops (51%). ¹H-NMR: 300 MHz, dmso-d₆ δ 3.83 (m, 2H); 3.63 (m, 2H); 3.59-3.23 (m, 3H); 3.15 (m, 2H); 2.16 (m, 2H).

### e) (R)-1,4-Diazabicyclo[3.2.1]octane dihydrochloride

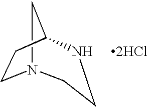

To a slowly stirring suspension of (R)-2-(2-chloro-ethyl)-piperazine dihydrochloride (1.47 g, 6.63 mmol) in 5 mL of water, was added a solution of NaOH (1.09 g, 27.18 mmol) in 5 mL of water. After 5 min, the aqueous solution was extracted three times with CHCl₃. The combined organic extracts were dried over MgSO₄, filtered and evaporated in vacuo to give an oil which was treated with 4 mL of conc. HCl to give a solution that was evaporated to dryness. The residue was dried under high vacuum to give 986 mg of the title compound as a white hygroscopic solid (80%). ¹H-NMR: 300 MHz, dmso-d₆ δ 4.28 (s, 1H); 3.75 (d, 1H); 3.66-3.3 (m, 7H); 2.33 (m, 2H).

### Intermediate 3: Lithium 5-phenyl-oxazole-2-carboxylate

### a) N-(2-Oxo-2-phenyl-ethyl)-oxalamic acid ethyl ester

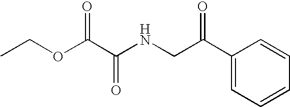

To a cooled (ice bath) mixture of 2-aminoacetophenone hydrochloride (2.64 g, 15.38 mmol) and ethyl chlorooxoacetate (1.81 mL, 16.15 mmol) in 50 mL of CH₂Cl₂ was added triethylamine (4.5 mL, 32.3 mmol). The resulting reaction mixture was stirred at room temperature for 72 hr. The mixture was then partitioned between CH₂Cl₂ and 1 N HCl. The layers were separated and the aqueous layer was extracted with CH₂Cl₂. The organic extracts were combined, dried over MgSO₄, filtered and concentrated in vacuo. ¹H-NMR analysis indicates product and cyclized oxazole present in approx. 9:1 ratio. The product was used without further purification.

¹H-NMR of amide: 300 MHz, CDCl₃ δ 8.05 (br s, 1H); 7.98 (m, 2H); 7.65 (m, 1H); 7.55 (m, 2H); 4.83 (d, 2H); 4.4 (quart., 2H); 1.42 (t, 3H).

### b) 5-Phenyl-oxazole-2-carboxylic acid ethyl ester

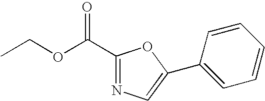

A solution of N-(2-Oxo-2-phenyl-ethyl)-oxalamic acid ethyl ester (approx. 15.3 mmol) in 15 mL of POCl₃ was heated at reflux for 3 hr. The volume was then reduced in vacuo and the residue was cautiously partitioned between CH₂Cl₂ and 5% aqueous Na₂CO₃. The layers were separated and the aqueous layer was extracted with CH₂Cl₂. The organic extracts were combined, dried over MgSO₄, filtered and concentrated in vacuo. The residue was chromatographed on silica gel (100% Hexane to 20% EtOAc/Hexane gradient) to give a pale amber solid weighing 2.44 g (11.23 mmol, 73% over two steps). ¹H-NMR: 300 MHz, CDCl₃ δ 7.76 (m, 2H); 7.52 (s, 1H); 7.45 (m, 3H); 4.5 (quart., 2H); 1.46 (t, 3H).

### c) Lithium 5-phenyl-oxazole-2-carboxylate

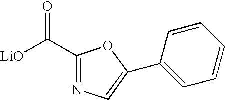

A solution of LiOH.H₂O (491 mg, 11.7 mmol) in 15 mL of water was added to a stirring solution of 5-phenyl-oxazole-2-carboxylic acid ethyl ester (2.42 g, 11.14 mmol) in 15 mL of THF. 3 mL of MeOH was added and the mixture was stirred overnight at room temp. The reaction mixture was then concentrated in vacuo and the resulting pale yellow solid was triturated with acetone. After removal of acetone and drying under high vacuum, a quantitative yield of the title compound as an off-white solid was obtained. LC/MS (APcI): (M+H)⁺=190.1.

### EXAMPLES

### Example 1

### (1,4-Diazabicyclo[3.2.1]oct-4-yl)-(5-pyridin-3-yl-thiophen-2-yl)-methanone

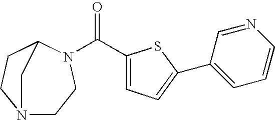

To a stirred solution of 5-(2-pyridyl)thiophene-2-carboxylic acid (45.0 mg, 0.22 mmol), O-(benzotriazol-1-yl)-N,N,N′,N′-tetramethyluronium tetrafluoroborate TBTU (71.0 mg, 0.22 mmol), and 1-hydroxybenzotriazole hydrate (30.0 mg, 0.22 mmol) in DMF (2 mL), was added diisopropylethylamine (0.05 mL, 0.29 mmol). After 5 min, a mixture of 1,4-diazabicyclo[3.2.1]octane dihydrochloride salt (40.0 mg, 0.22 mmol) and 0.1 mL DIEA (0.1 mL, 0.59 mmol) in DMF (1 mL) was added. The reaction mixture was stirred at room temperature overnight. The reaction mixture was then partitioned between EtOAc and 5% Na₂CO₃. The layers were separated and the aqueous phase was extracted with EtOAc. The organic extracts were combined, dried over MgSO₄, filtered and concentrated in vacuo. The residue was chromatographed on silica gel using a gradient of 100:0 to 95:5 CHCl₃:MeOH. The product was obtained as an off-white solid (39 mg, 60%). MS (APCI+) 300 [M+1]+. ¹H NMR (300.132 MHz, CDCl₃) δ 8.89 (s, 1H), 8.58 (d, J=4.2 Hz, 1H), 7.87 (dt, J=8.0 Hz, J=1.9 Hz, 1H), 7.34 (dd, J=3.1 Hz, J=4.9 Hz, 1H), 7.30 (q, J=7.9 Hz, 2H), 5.04 (s, 1H), 4.11 (dd, J=13.9 Hz, J=5.2 Hz, 1H), 3.43 (t, J=10.7 Hz, 1H), 3.23-3.04 (m, 2H), 2.88 (dd, J=4.7 Hz, J=13.8 Hz, 1H), 2.77 (d, J=11.0 Hz, 1H), 2.55-2.34 (m, 2H), 2.19-1.97 (m, 2H).

### Example 2

### (1,4-Diazabicyclo[3.2.1]oct-4-yl)-(5-phenyl-thiophen-2-yl)-methanone

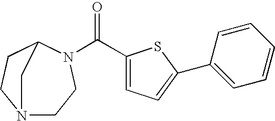

By the process described in Example 1, diazabicyclo[3.2.1]octane dihydrochloride salt with 5-phenyl-thiophene-2-carboxylic acid to afford the title compound as an amber gum. MS (APCI+) 299 [M+1]⁺. ¹H NMR (300.132 MHz, CDCl₃) δ 7.61 (dt, J=7.5, 1.7 Hz, 2H), 7.40 (tt, J=7.3, 1.6 Hz, 2H), 7.34 (dt, J=7.2, 1.5 Hz, 1H), 7.28 (d, J=3.6 Hz, 1H), 7.23 (d, J=3.6 Hz, 1H), 4.98 (m, 1H), 4.03 (dd, J=13.5, 4.8 Hz, 1H), 3.37 (m, 1H), 3.09 (d, J=13.1 Hz, 1H), 3.05 (t, J=8.0 Hz, 3H), 2.77 (dd, J=13.4, 4.2 Hz, 1H), 2.63 (d, J=11.6 Hz, 1H), 2.06-1.97 (m, 2H).

### Example 3

### [5-(4-Chloro-phenyl)-furan-2-yl]-(1,4-diazabicyclo[3.2.1]oct-4-yl)-methanone

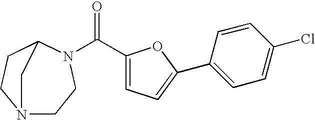

By the process described in Example 1, diazabicyclo[3.2.1]octane dihydrochloride salt was reacted with 5-(4-chloro-phenyl)-furan-2-carboxylic acid to afford the title compound as a gum. MS (APCI+) 317/319 [M+1]⁺. ¹H NMR (300.132 MHz, CDCl₃) δ 7.61 (d, J=8.6 Hz, 2H), 7.38 (d, J=8.6 Hz, 2H), 7.06 (d, J=3.5 Hz, 1H), 6.71 (d, J=3.5 Hz, 1H), 5.08 (m, 1H), 4.13 (dd, J=13.8 Hz, J=5.3 Hz, 1H), 3.77-3.22 (m, 1H), 3.06 (t, J=7.5 Hz, 4H), 2.79 (d, J=10.0 Hz, 1H), 2.66 (d, J=9.9 Hz, 1H), 2.04 (t, J=6.5 Hz, 2H).

### Example 4

### (1,4-Diazabicyclo[3.2.1]oct-4-yl)-(5-phenyl-furan-2-yl)-methanone

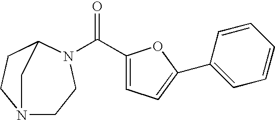

By the process described in Example 1, diazabicyclo[3.2.1]octane dihydrochloride salt was reacted with 5-phenyl-furan-2-carboxylic acid to afford the title compound as a white solid. MS (APCI+) 283 [M+1]+. ¹H NMR (300.132 MHz, CDCl₃) δ 7.68-7.62 (m, 2H), 7.50-7.32 (m, 4H), 6.77 (bs, 1H), 5.56 (m, 1H), 4.72 (m, 1H), 3.72 (m, 2H), 3.38 (m, 5H), 2.61-2.43 (m, 1H), 2.38-2.20 (m, 1H).

### Example 5

### Benzofuran-2-yl-(1,4-diazabicyclo[3.2.1]oct-4-yl)-methanone

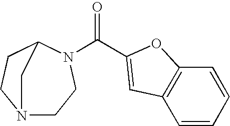

By the process described in Example 1, diazabicyclo[3.2.1]octane dihydrochloride salt was reacted with 2-benzofurancarboxylic acid to afford the title compound as an off-white solid (34 mg, 60%). MS (APCI+) 257 [M+1]⁺. ¹H NMR (300.132 MHz, CDCl₃) δ 7.68 (d, J=7.7 Hz, 1H), 7.53 (d, J=8.3 Hz, 1H), 7.44 (t, J=6.5 Hz, 1H), 7.42 (s, 1H), 7.32 (t, J=7.5 Hz, 1H), 5.39 (s, 1H), 4.49 (dd, J=4.5 Hz, J=14.4 Hz, 1H), 3.67 (quintet, J=6.7 Hz, 1H), 3.53 (sextet, J=6.0 Hz, 1H), 3.44-3.02 (m, 4H), 2.42-2.14 (m, 2H), 1.61-1.54 (m, 1H).

### Example 6

### (1,4-Diazabicyclo[3.2.1]oct-4-yl)-(1-methyl-1H-indol-2-yl)-methanone

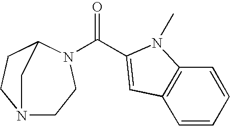

By the process described in Example 1, diazabicyclo[3.2.1]octane dihydrochloride salt was reacted with 3H-indole-2-carboxylic acid to afford the title compound as a colorless gum. MS (APCI+) 270 [M+1]⁺. ¹H NMR (300.132 MHz, CDCl₃) δ 7.62 (d, J=8.0 Hz, 1H), 7.35 (t, J=8.9 Hz, 1H), 7.28 (d, J=8.0 Hz, 1H), 7.14 (t, J=7.5 Hz, 1H), 6.59 (s, 1H), 5.32-4.67 (m, 1H), 4.31-3.78 (m, 1H), 3.84 (s, 3H), 3.08 (d, J=7.7 Hz, 1H), 3.05 (t, J=7.2 Hz, 3H), 2.88-2.71 (m, 1H), 2.69-2.54 (m, 1H), 1.99 (m, 2H).

### Example 7

### Biphenyl-3-yl-(1,4-diazabicyclo[3.2.1]oct-4-yl)-methanone

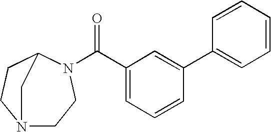

By the process described in Example 1, diazabicyclo[3.2.1]octane dihydrochloride salt with biphenyl-3-carboxylic acid to afford the title compound as a gum. MS (APCI+) 293 [M+1]+. ¹H NMR (300.132 MHz, CDCl₃) δ 7.68-7.56 (m, 4H), 7.49 (d, J=8.0 Hz, 1H), 7.45 (t, J=7.4 Hz, 2H), 7.37 (t, J=7.5 Hz, 2H), 5.24 (bs, 1H), 5.24 (bs, 1H), 3.41 (bs, 1H), 3.13-2.95 (m, 4H), 2.95-2.43 (m, 2H), 2.18-1.68 (m, 2H).

### Example 8

### (1,4-Diazabicyclo[3.2.1]oct-4-yl)-(4-methoxy-phenyl)-methanone

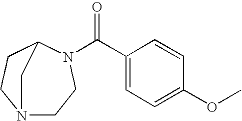

By the process described in Example 1, diazabicyclo[3.2.1]octane dihydrochloride salt was reacted with 4-methoxy-benzoic acid to afford the title compound as an off-white film. MS (APCI+) 247 [M+1]+. ¹H NMR (300.132 MHz, CDCl₃) δ 7.38 (d, J=8.5 Hz, 2H), 6.92 (d, J=8.7 Hz, 2H), 3.84 (s, 3H), 3.29 (m, 1H), 3.07 (d, J=10.2 Hz, 4H), 2.86-2.73 (m, 2H), 2.66 (d, J=10.7 Hz, 2H), 2.00 (m, 2H).

### Example 9

### (1,4-Diazabicyclo[3.2.1]oct-4-yl)-(1H-indol-5-yl)-methanone

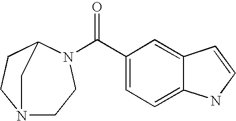

By the process described in Example 1, diazabicyclo[3.2.1]octane dihydrochloride salt was reacted with 3H-indole-5-carboxylic acid to afford the title compound as an off-white film. MS (APCI+) 256 [M+1]+. ¹H NMR (300.132 MHz, CDCl₃) δ 8.36 (bs, 1H), 7.72 (s, 1H), 7.41 (d, J=8.4 Hz, 1H), 7.26 (m, 1H), 6.60 (s, 1H), 3.70-3.16 (m, 1H), 3.09 (d, J=12.5 Hz, 2H), 3.04 (t, J=8.0 Hz, 2H), 2.85-2.66 (m, 1H), 2.66-2.52 (m, 1H), 1.98 (m, 2H), 1.70 (m, 2H).

### Example 10

### (1,4-Diazabicyclo[3.2.1]oct-4-yl)-naphthalen-2-yl-methanone

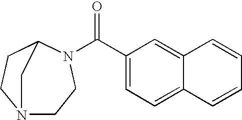

By the process described in Example 1, diazabicyclo[3.2.1]octane dihydrochloride salt was reacted with naphthalene-2-carboxylic acid to afford the title compound as an amber gum. MS (APCI+) 267 [M+1]+. ¹H NMR (300.132 MHz, CDCl₃) δ 7.88 (d, J=8.0 Hz, 2H), 7.87 (t, J=6.5 Hz, 2H), 7.54 (m, 2H), 7.48 (dd, J=8.3, 1.3 Hz, 1H), 5.26 (m, 1H), 4.30 (m, 1H), 3.43 (m, 1H), 3.09 (d, J=12.0 Hz, 2H), 3.05 (m, 2H), 2.88 (m, 1H), 2.75-2.46 (m, 1H), 2.00 (s, 2H).

### Example 11

### 4-[5-((R)-1,4-Diaza-bicyclo[3.2.1]octane-4-carbonyl)-thiophen-2-yl]-N,N-dimethyl-benzamide

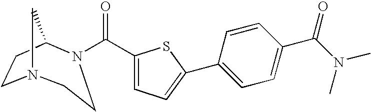

### a) 4-(5-Bromo-thiophen-2-yl)-N,N-dimethyl-benzamide

4-(N,N-Dimethylaminocarbonyl)phenylboronic acid (415 mg, 2.15 mmole), 2-5-dibromothiophene (1.14 grams, 4.73 mmole), cesium carbonate (2.1 grams, 6.45 mmole), and tetrakis(triphenylphosphine)palladium (240 mg, 0.22 mmole) were slurried in ethylene glycol dimethyl ether/water/ethanol (7:3:2, 20 ml). The mixture was heated in a round bottom flask at 80° C. overnight. The mixture was cooled, treated with water and extracted with chloroform (3 times). The organic layers were combined, dried over sodium sulfate, filtered, and evaporated under reduced pressure to afford the product as an oil. The oil was purified on silica gel using 40% ethyl acetate in hexanes as the eluant. The compound was obtained as a pale yellow solid (59% recovery). ¹H NMR (300.132 MHz, DMSO) δ 7.67 (d, J=9.4 Hz, 1H), 7.46-7.43 (m, 3H), 7.29 (d, J=4.7 Hz, 1H), 2.95 (s, 6H); MS m/z: 311 (M+H)⁺.

### b) 5-(4-Dimethylcarbamoyl-phenyl)-thiophene-2-carboxylic acid ethyl ester

4-(5-Bromo-thiophen-2-yl)-N,N-dimethyl-benzamide (155 mg, 0.50 mmole), palladium bistriphenylphosphine dichloride (18 mg, 0.025 mmole), and triethylamine (119 mg, 1.18 mmole) were taken up in ethanol (2 mL) in a 8 ml endeavor reaction tube. The solution was then taken up to 20 atm of carbon monoxide and heated to 100° C. for 24 hours. The solution was cooled filtered through diatomaceous earth washing with ethanol. The resultant mother liquor was concentrated under reduced pressure to an oil. The oil was purified on silica gel using 35% ethyl acetate in hexanes as the eluant. The compound was obtained as a pale yellow solid (84% recovery). ¹H NMR (300.132 MHz, DMSO) δ 7.85-7.78 (m, 3H), 7.67 (d, J=4.0 Hz, 1H), 7.49 (d, J=8.2 Hz, 2H), 4.32 (q, J=7.1 Hz, 2H), 2.96 (s, 6H), 1.32 (t, J=7.1 Hz, 3H); MS m/z: 304 (M+H)⁺.

### c) Lithium 5-(4-dimethylcarbamoyl-phenyl)-thiophene-2-carboxylate

5-(4-Dimethylcarbamoyl-phenyl)-thiophene-2-carboxylic acid ethyl ester was taken up in tetrahydrofuran/methanol/water (1:1:1, 6 ml) and lithium hydroxide (19 mg, 0.45 mmole) was added and the solution was stirred overnight at room temperature. The entire mixture was evaporated under reduced pressure to afford the product as a white solid (100% recovery). MS m/z: 276 (M+H)⁺.

d) (5R)-1,4-Diaza-bicyclo[3.2.1]octane (102 mg, 0.55 mmole), lithium 5-(4-dimethylcarbamoyl-phenyl)-thiophene-2-carboxylate (152 mg, 0.55 mmole), 2(1H-benzotriazole-1-yl)-1,1,3,3-tetramethyluronium tetrafluoroborate (177 mg, 0.55 mmole), 1-hydroxybenzotriazole (74 mg, 0.55 mmole) and diisopropylethylamine (223 mg, 1.72 mmole) were dissolved in N,N-dimethylformamide (5 mL) and stirred at room temperature overnight. The solution was treated with 1 N sodium hydroxide and extracted with chloroform (3 times). The organic layers were combined, dried over sodium sulfate, filtered, and evaporated under reduced pressure to afford the product as an oil. The material was purified by silica gel using 5% 7 N ammoniated methanol in chloroform. The title compound was obtained as a tan solid (22% recovery). ¹H NMR (300.132 MHz, DMSO) δ 7.76 (d, J=7.0 Hz, 2H), 7.57 (s, 1H), 7.49-7.42 (m, 3H), 4.79 (s, 1H), 3.84-3.75 (m, 1H), 2.96 (s, 6H), 2.89-2.76 (m, 5H), 2.69-2.55 (m, 1H), 2.46-2.43 (m, 1H), 1.93 (s, 2H); MS m/z: 370 (M+H)⁺.

The following compounds were synthesized in a fashion analogous to Example 11.

### Example 12

### 3-[5-((R)-1,4-Diaza-bicyclo[3.2.1]octane-4-carbonyl)-thiophen-2-yl]-N,N-dimethyl-benzamide

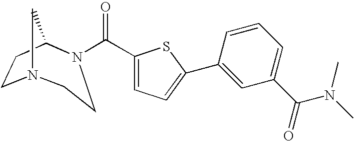

By a process analogous to that of Example 1, the title compound was obtained as a tan solid in 22% yield. ¹H NMR (300.132 MHz, DMSO) δ 7.78-7.67 (m, 2H), 7.61-7.45 (m, 2H), 7.43-7.34 (m, 2H), 4.79 (s, 1H), 3.86-3.75 (m, 2H), 3.01-2.77 (m, 8H), 2.69-2.55 (m, 2H), 2.47-2.43 (m, 2H), 1.96-1.90 (m, 2H); MS m/z: 370 (M+H)⁺.

### Example 13

### (R)-1,4-Diaza-bicyclo[3.2.1]oct-4-yl-(5-phenyl-oxazol-2-yl)-methanone hydrochloride

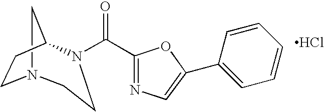

DMF (6 mL) was added to a reaction flask containing lithium 5-phenyl-oxazole-2-carboxylate (232 mg, 1.19 mmol), TBTU (369 mg, 1.15 mmol) and HOBt (155 mg, 1.15 mmol). In a separate vial, (R)-1,4-diazabicyclo[3.2.1]octane dihydrochloride (200 mg, 1.08 mmol) and diisopropylethylamine (0.59 mL, 3.4 mmol) were mixed in DMF (7 mL) to give a solution which was added to the reaction flask. The resulting reaction mixture was stirred overnight at room temp. The mixture was then partitioned between EtOAc and 1 N NaOH. The layers were separated and the aqueous layer was extracted with EtOAc. The organic extracts were combined, dried over MgSO₄, filtered and concentrated in vacuo. The residue was chromatographed on silica gel (100% CHCl₃ to 3% MeOH (containing 7 N N₃) in CHCl₃) to give a colorless viscous oil as the free base product. The oil was dissolved in 2 mL of CHCl₃ and 20 mL of diethyl ether was added. Approx. 0.5 mL of 4 N HCl in dioxane were added and the resulting precipitate was collected by vacuum filtration. 253 mg of the title compound was obtained as a white solid. ¹H-NMR: 300 MHz, room temperature, dmso-d₆ δ 11.5 (br s, 1H); 7.96 (s, 1H); 7.8 (m, 2H); 7.5 (m, 3H); 5.88, 5.33 (2 br s, 1H); 5.02, 4.43 (2 m, 1H); 3.9-3.2 (m, 7H); 2.4 (m, 1H), 2.21 (m, 1H). ¹H-NMR: 300 MHz, 90° C., dmso-d₆ δ 7.78 (m, 3H); 7.5 (m, 3H); 5.6 (br m, 1H); 4.7 (br m, 1H); 3.8-3.2 (m, 7H); 2.46 (m, 1H); 2.25 (m, 1H). LC/MS (APcI): (M+H)⁺=284.1.

### Example 14

### (R)-1,4-Diaza-bicyclo[3.2.1]oct-4-yl-(5-pyridin-3-yl-oxazol-2-yl)-methanone dihydrochloride

### a) (2-Oxo-2-pyridin-3-yl-ethyl)-carbamic acid tert-butyl ester

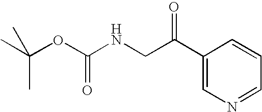

To a solution of 3-bromopyridine (1.21 mL, 12.6 mmol) in 15 mL of dry THF was added isopropylmagnesium chloride (2 M in THF, 6.3 mL, 12.6 mmol) at room temperature under N₂. After 45 min., in a separate flask, isopropylmagnesium chloride (4.9 mL, 9.8 mmol) was added to a cooled (−15 to −10° C.) slurry of N-(tert-butoxycarbonyl)glycine N′-methoxy-N′-methylamide (2.18 g, 10.0 mmol) in 15 mL of dry THF under N₂. After the Br—Mg exchange reaction had stirred for a total of 1 hr, the resulting mixture was added to the Weinreb amide anion solution. After the entire contents had been added, the reaction mixture was allowed to warm to room temperature and stirred overnight. The mixture was then partitioned between EtOAc and water. The layers were separated and the aqueous layer was extracted with EtOAc. The organic extracts were combined, dried over MgSO₄, filtered and concentrated in vacuo. The residue was chromatographed on silica gel (100% Hexane to 25% EtOAc/Hexane gradient) to give 1.57 g of a white solid as desired product (66%). ¹H-NMR: 300 MHz, CDCl₃ δ 9.17 (m, 1H); 8.82 (m, 1H); 8.23 (m, 1H); 7.44 (m, 1H); 5.45 (br s, 1H); 4.66 (d, 2H); 1.48 (s, 9H).

### b) 2-Amino-1-pyridin-3-yl-ethanone dihydrochloride

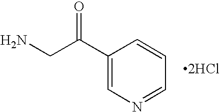

To a solution of (2-oxo-2-pyridin-3-yl-ethyl)-carbamic acid tert-butyl ester in MeOH (7 mL) was added 5-6N HCl in 2-propanol (7 mL). The mixture was heated at 50° C. for 2 hr, then concentrated in vacuo and dried on high vacuum. A quantitative yield of off-white solid was obtained and used without further purification.

### c) N-(2-Oxo-2-pyridin-3-yl-ethyl)-oxalamic acid ethyl ester

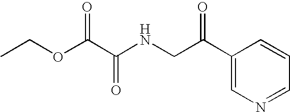

To a cooled (ice bath) mixture of 2-amino-1-pyridin-3-yl-ethanone dihydrochloride (913 mg, 4.37 mmol) and ethyl chlorooxoacetate (0.54 mL, 4.8 mmol) in 15 mL of CH₂Cl₂ was added triethylamine (1.9 mL, 13.6 mmol). The resulting reaction mixture was stirred at room temperature overnight. The mixture was then partitioned between CH₂Cl₂ and water. The layers were separated and the aqueous layer was extracted with CH₂Cl₂. The organic extracts were combined, dried over MgSO₄, filtered and concentrated in vacuo. The residue was purified by column chromatography on silica gel (20% EtOAc in Hexane to 80% EtOAc in Hexane gradient). LC/MS (APcI): (M+H)⁺=237.1

### d) 5-Pyridin-3-yl-oxazole-2-carboxylic acid ethyl ester

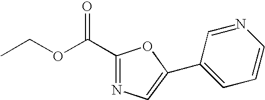

To a cooled (ice bath) mixture of N-(2-Oxo-2-pyridin-3-yl-ethyl)-oxalamic acid ethyl ester (750 mg, 3.18 mmol), triphenylphosphine (1.89 g, 7.21 mmol), and hexachloroethane (1.55 g, 6.55 mmol) in 30 mL CH₂Cl₂ was added triethylamine (1.67 mL, 11.96 mmol). The reaction mixture was stirred for 1 hr, then chromatographed on silica gel (EtOAc/Hexane gradient). 700 mg of an off-white solid were collected corresponding to desired oxazole and containing a small amount of triphenylphosphine oxide. ¹H-NMR: 300 MHz, CDCl₃ δ 9.02 (s, 1H); 8.65 (m, 1H); 8.06 (m, 1H); 7.62 (s, 1H); 7.42 (m, 1H); 4.51 (quart, 2H); 1.47 (t, 3H). LC/MS (APcI): (M+H)⁺=219.1.

### e) Lithium 5-pyridin-3-yl-oxazole-2-carboxylate

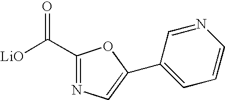

A solution of LiOH.H₂O (133 mg, 3.18 mmol) in 7 mL of water was added to a stirring solution of 5-pyridin-3-yl-oxazole-2-carboxylic acid ethyl ester (700 mg, 3.18 mmol) in 7 mL of THF. 1 mL of MeOH was added and the mixture was stirred overnight at room temp. The reaction mixture was then concentrated in vacuo and the resulting pale yellow solid was triturated with acetone. After removal of acetone and drying under high vacuum, 530 mg of an off-white solid was obtained.

### f) (R)-1,4-Diaza-bicyclo[3.2.1]oct-4-yl-(5-pyridin-3-yl-oxazol-2-yl)-methanone dihydrochloride

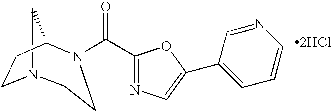

DMF (6 mL) was added to a reaction flask containing lithium 5-pyridin-3-yl-oxazole-2-carboxylate (530 mg, 2.7 mmol), TBTU (867 mg, 2.7 mmol) and HOBt (365 mg, 2.7 mmol). In a separate vial, (R)-1,4-diazabicyclo[3.2.1]octane dihydrochloride (500 mg, 2.7 mmol) and diisopropylethylamine (1.41 mL, 8.1 mmol) were mixed in DMF (7 mL) to give a solution which was added to the reaction flask. The resulting reaction mixture was stirred overnight at room temperature, then concentrated in vacuo. The residue was chromatographed on silica gel (100% CHCl₃ to 3% MeOH (containing 7N N₃) in CHCl₃) to give a colorless viscous oil as the free base product. The oil was dissolved in 2 mL of CHCl₃ and 20 mL of diethyl ether was added. Approx. 2 mL of 4 N HCl in dioxane were added and the resulting precipitate was collected by vacuum filtration. 495 mg of the title compound was obtained as a white hygroscopic solid. ¹H-NMR: 300 MHz, room temperature, dmso-d₆ δ 11.0 (br s, 1H); 9.08 (s, 1H); 8.68 (d, 1H); 8.25 (d, 1H); 8.12 (s, 1H); 7.64 (m, 1H); 5.83, 5.34 (2 br s, 1H); 4.97, 4.42 (2 m, 1H); 4.0-3.2 (m, 7H); 2.4 (m, 1H), 2.24 (m, 1H). LC/MS (APcI): (M+H)⁺=285.2.

### Example 15

### (R)-1,4-Diaza-bicyclo[3.2.1]oct-4-yl-(5-pyridin-4-yl-oxazol-2-yl)-methanone

### a) (2-Oxo-2-pyridin-4-yl-ethyl)-carbamic acid tert-butyl ester

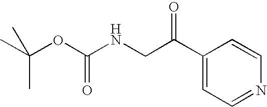

4-Bromopyridine hydrochloride (2.45 g, 12.6 mmol) was treated with 65 mL of 5% aqueous Na₂CO₃ and extracted twice with 30 mL Et₂O. The ethereal extracts were dried over MgSO₄, filtered and the solvent was removed in vacuo. The residue was immediately dissolved in dry THF and isopropylmagnesium chloride (2 M in THF, 6.3 mL, 12.6 mmol) was added at room temp under N₂. After 45 min., in a separate flask, isopropylmagnesium chloride (4.9 mL, 9.8 mmol) was added to a cooled (−15 to −10° C.) slurry of N-(tert-butoxycarbonyl)glycine N′-methoxy-N′-methylamide (2.18 g, 10.0 mmol) in 15 mL of dry THF under N₂. After the Br—Mg exchange reaction had stirred for a total of 1 hr, the resulting mixture was added to the Weinreb amide anion solution. After the entire contents had been added, the reaction mixture was allowed to warm to room temperature and stirred overnight. The mixture was then partitioned between EtOAc and water. The layers were separated and the aqueous layer was extracted with EtOAc. The organic extracts were combined, dried over MgSO₄, filtered and concentrated in vacuo. The residue was chromatographed on silica gel (100% Hexane to 30% EtOAc/Hexane gradient) to give 1.2 g of an amber solid as desired product. ¹H-NMR: 300 MHz, CDCl₃ δ 8.67 (d, 1H); 8.04 (d, 1H); 7.85 (m, 1H); 7.5 (m, 1H); 5.36 (br s, 1H); 4.88 (d, 2H); 1.48 (s, 9H).

### b) 2-Amino-1-pyridin-4-yl-ethanone dihydrochloride

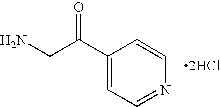

To a solution of (2-Oxo-2-pyridin-4-yl-ethyl)-carbamic acid tert-butyl ester in MeOH (7 mL) was added 5-6 N HCl in 2-propanol (7 mL). The mixture was heated at 50° C. for 2 hr, then concentrated in vacuo and dried on high vacuum. The product was obtained in quantitative yield of off-white solid and used without further purification.

### c) 5-Pyridin-4-yl-oxazole-2-carboxylic acid ethyl ester

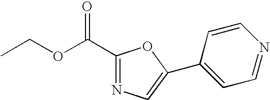

To a cooled (ice bath) mixture of 2-amino-1-pyridin-3-yl-ethanone dihydrochloride (5.08 mmol) and ethyl chlorooxoacetate (0.62 mL, 5.5 mmol) in 20 mL of CH₂Cl₂ was added triethylamine (2.26 mL, 16.25 mmol). The resulting reaction mixture was stirred at room temperature overnight. The mixture was then partitioned between CH₂Cl₂ and water. The layers were separated and the aqueous layer was extracted with CH₂Cl₂. The organic extracts were combined, dried over MgSO₄, filtered and concentrated in vacuo. LC/MS (APcI): (M+H)⁺=219.1 corresponding to cyclized oxazole was observed as the major component in the product mixture. A smaller peak corresponding to the uncyclized amide ((M+H)⁺=237.1) was also observed. The mixture was chromatographed on silica gel (100% Hexane to 35% EtOAc in Hexane gradient) to give 142 mg of oxazole product (13%).

### d) Lithium 5-pyridin-4-yl-oxazole-2-carboxylate

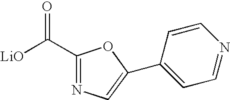

A solution of LiOH.H₂O (30 mg, 0.17 mmol) in 3 mL of water was added to a stirring solution of 5-pyridin-4-yl-oxazole-2-carboxylic acid ethyl ester (140 mg, 0.64 mmol) in 3 mL of THF. 0.5 mL of MeOH was added and the mixture was stirred overnight at room temp. The reaction mixture was then concentrated in vacuo and the resulting pale yellow solid was triturated with acetone. After removal of acetone and drying under high vacuum, quantitative yield of an off-white solid was obtained. ¹H-NMR: 300 MHz, dmso-d₆ δ 8.62 (d, 2H); 7.83 (s, 1H); 7.64 (d, 2H). LC/MS (APcI): (M+H)⁺=191.1.

### e) (R)-1,4-Diaza-bicyclo[3.2.1]oct-4-yl-(5-pyridin-4-yl-oxazol-2-yl)-methanone dihydrochloride

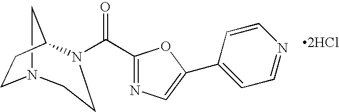

DMF (3 mL) was added to a reaction flask containing lithium 5-pyridin-4-yl-oxazole-2-carboxylate (60 mg, 0.3 mmol), TBTU (87 mg, 0.27 mmol) and HOBt (36 mg, 0.27 mmol). In a separate vial, (R)-1,4-diazabicyclo[3.2.1]octane dihydrochloride (50 mg, 0.27 mmol) and diisopropylethylamine (0.16 mL, 0.9 mmol) were mixed in DMF (2 mL) to give a solution which was added to the reaction flask. The resulting reaction mixture was stirred overnight at room temperature, then concentrated in vacuo. The residue was chromatographed on silica gel (100% CHCl₃ to 4% MeOH (containing 7 N N₃) in CHCl₃) to give a colorless viscous oil as the free base product. The oil was dissolved in 1 mL of CHCl₃ and 10 mL of diethyl ether was added. Approx. 0.5 mL of 4 N HCl in dioxane were added and the resulting precipitate was collected by vacuum filtration. 14 mg of the title compound as an off-white hygroscopic solid were obtained. ¹H-NMR: 300 MHz, room temperature, dmso-d₆ δ 11.38 (br s, 1H); 8.87 (d, 2H); 8.44 (s, 1H); 8.05 (d, 2H); 5.76, 5.33 (2 br s, 1H); 4.93, 4.43 (2 m, 1H); 4.0-3.2 (m, 7H); 2.4 (m, 1H), 2.24 (m, 1H). LC/MS (APcI): (M+H)⁺=285.1.

1. A compound according to Formula I: 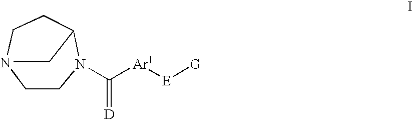
wherein:
D is selected from oxygen, sulfur or N(R¹)₂;
Ar¹ is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;
E is a single bond, —O, —S, or —NR¹;
G is selected from hydrogen, C₁-C₄alkoxy or Ar², where Ar² is a 5- or 6-membered aromatic or heteroaromatic ring having 0, 1 or 2 nitrogen atoms, 0 or 1 oxygen atoms, and 0 or 1 sulfur atoms;
where each Ar¹ or Ar² moiety independently is unsubstituted or has 1, 2 or 3 substituents selected from —R³, —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;
R¹, R¹ and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or
R² and R³ in combination is —(CH₂)ⱼG(CH₂)ₖ— wherein G is oxygen, sulfur, NR⁴, or a bond;
j is 2, 3 or 4;
k is 0, 1 or 2;
n is 0, 1 or 2, and
R⁴ is independently selected at each occurrence from hydrogen, —C₁-C₄alkyl, aryl, or heteroaryl,
and stereoisomers, enantiomers, in vivo-hydrolysable precursors or a pharmaceutically-acceptable salt thereof.

2. A compound according to claim 1, wherein:
D is oxygen;
Ar¹ is selected from phenyl or a 5-membered heteroaromatic ring having 0 or 1 nitrogen atoms, 0 or 1 oxygen atoms, and 0 or 1 sulfur atoms, or selected from a 9-membered fused aromatic or heteroaromatic ring system having 0, 1, 2 or 3 nitrogen atoms, 0 or 1 oxygen atoms, and 0 or 1 sulfur atoms;
wherein:
E is a single bond;
G is selected from hydrogen, methoxy or Ar², where Ar² is selected from a 6-membered aromatic or heteroaromatic ring having 0 or 1 nitrogen atoms, 0 or 1 oxygen atoms, and 0 or 1 sulfur atoms;
where each Ar¹ or Ar² moiety independently is unsubstituted or has 1, 2 or 3 substituents selected from halogen, —CN, —NO₂, —CF₃, —CH₃ or —C₂H₅;
and stereoisomers, enantiomers, in vivo-hydrolysable precursors or a pharmaceutically-acceptable salt thereof.

3. A compound according to claim 1, wherein:
D is oxygen;
Ar¹ is selected from phenyl, furanyl, thiophenyl or 1-methyl-1H-pyrrolyl:
E is a single bond;
G is selected from hydrogen, methoxy, phenyl or pyridyl, and
Ar¹ bears 1 halogen substituent;
and stereoisomers, enantiomers, in vivo-hydrolysable precursors or a pharmaceutically-acceptable salt thereof.

4. A compound according to claim 1, wherein:
E represents a single bond; or an enantiomer thereof, or a pharmaceutically-acceptable salt thereof.

5. A compound according to claim 1, wherein:
Ar¹ is furanyl or thiophenyl having optional substituents as defined herein.

6. A compound according to claim 1, selected from:
(1,4-diazabicyclo[3.2.1]oct-4-yl)-(5-pyridin-3-yl-thiophen-2-yl)-methanone;
(1,4-diazabicyclo[3.2.1]oct-4-yl)-(5-phenyl-thiophen-2-yl)-methanone;
[5-(4-chloro-phenyl)-furan-2-yl]-(1,4-diazabicyclo[3.2.1]oct-4-yl)-methanone;
(1,4-diazabicyclo[3.2.1]oct-4-yl)-(5-phenyl-furan-2-yl)-methanone;
benzofuran-2-yl-(1,4-diazabicyclo[3.2.1]oct-4-yl)-methanone;
(1,4-diazabicyclo[3.2.1]oct-4-yl)-(1-methyl-1H-indol-2-yl)-methanone;
biphenyl-3-yl-(1,4-diazabicyclo[3.2.1]oct-4-yl)-methanone;
(1,4-diazabicyclo[3.2.1]oct-4-yl)-(4-methoxy-phenyl)-methanone;
(1,4-diazabicyclo[3.2.1]oct-4-yl)-(1H-indol-5-yl)-methanone;
(1,4-diazabicyclo[3.2.1]oct-4-yl)-naphthalen-2-yl-methanone;
4-[5-((R)-1,4-diaza-bicyclo[3.2.1]octane-4-carbonyl)-thiophen-2-yl]-N,N-dimethyl-benzamide;
3-[5-((R)-1,4-diaza-bicyclo[3.2.1]octane-4-carbonyl)-thiophen-2-yl]-N,N-dimethyl-benzamide;
(R)-1,4-diaza-bicyclo[3.2.1]oct-4-yl-(5-phenyl-oxazol-2-yl)-methanone hydrochloride;
(R)-1,4-diaza-bicyclo[3.2.1]oct-4-yl-(5-pyridin-3-yl-oxazol-2-yl)-methanone dihydrochloride, or
(R)-1,4-diaza-bicyclo [3.2.1]oct-4-yl-(5-pyridin-4-yl-oxazol-2-yl)-methanone, or stereoisomers, enantiomers, in vivo-hydrolysable precursors or a pharmaceutically-acceptable salt thereof.

7. A method of treatment or prophylaxis of a disease or condition in which activation of the α7 nicotinic receptor is beneficial which method comprises administering a therapeutically-effective amount of a compound according to claim 1 to a subject suffering from said disease or condition.

8. The method of claim 7, wherein said disease or condition is anxiety, schizophrenia, mania or manic depression.

9. A method of treatment or prophylaxis of neurological disorders, psychotic disorders or intellectual impairment disorders, which comprises administering a therapeutically effective amount of a compound according to claim 1.

10. The method of claim 9, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

11. A method for inducing the cessation of smoking comprising administering an effective amount of a compound according to claim 1.

12. A pharmaceutical composition comprising a compound according to claim 1 and a pharmaceutically-acceptable diluent, lubricant or carrier.

13. A method of treatment or prophylaxis of a disease or condition in which activation of the α7 nicotinic receptor is beneficial which method comprises administering a therapeutically-effective amount of a pharmaceutical composition according to claim 12 to a subject suffering from said disease or condition.

14. The method of claim 13, wherein said disease or condition is anxiety, schizophrenia, mania or manic depression.

15. A method of treatment or prophylaxis of neurological disorders, psychotic disorders or intellectual impairment disorders, which comprises administering a therapeutically effective amount of a pharmaceutical composition according to claim 12.

16. The method of claim 10, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

17. A method for inducing the cessation of smoking comprising administering an effective amount of a pharmaceutical composition according to claim 12.

18. The use of a compound according to claim 1, an enantiomer thereof or a pharmaceutically-acceptable salt thereof, in the manufacture of a medicament for the treatment or prophylaxis of human diseases or conditions in which activation of the α7 nicotinic receptor is beneficial selected from neurological disorders, psychotic disorders, intellectual impairment disorders, Alzheimer's disease, learning deficit, cognition deficit, attention deficit, memory loss, Attention Deficit Hyperactivity Disorder, anxiety, schizophrenia, mania or manic depression, Parkinson's disease, Huntington's disease, Tourette's syndrome, or neurodegenerative disorders in which there is loss of cholinergic synapses.

19. The use of a compound according to claim 1, in the manufacture of a medicament for the treatment or prophylaxis of jetlag, pain, or ulcerative colitis or to facilitate the cessation of smoking or the treatment of nicotine addiction or craving including that resulting from exposure to products containing nicotine.
